(19) United States
(12) Patent Application Publication
(10) Pub. No.: US 2025/0025593 A1
(43) Pub. Date: Jan. 23, 2025

(54) PLASMA-ACTIVATED GAS DISINFECTION DEVICE AND METHOD

(71) Applicant: Xi’an Jiaotong University, Xi'an (CN)

(72) Inventors: Dingxin LIU, Xi'an (CN); Zifeng WANG, Xi'an (CN); Yue FENG, Xi'an (CN); Luge WANG, Xi'an (CN); Zizhu ZHANG, Xi'an (CN); Xiangyu WANG, Xi'an (CN); Xiaohua WANG, Xi'an (CN); Mingzhe RONG, Xi'an (CN)

(73) Assignee: Xi’an Jiaotong University, Xi'an (CN)

(21) Appl. No.: 18/469,853

(22) Filed: Sep. 19, 2023

(30) Foreign Application Priority Data
Jul. 17, 2023 (CN) 202310875786.0

(51) Int. Cl. A61L 9/16; H01J 37/32

(52) U.S. Cl. CPC A61L 9/16; H01J 37/32348; H01J 37/32844; A61L 2209/11

(57) ABSTRACT

A plasma-activated gas disinfection device includes a plasma generating unit, a disinfection chamber, an air pump and a fluid control module, where the plasma generating unit works in a first working mode during a first time period, and generates a first plasma active gas in the first working mode; the plasma generating unit works in a second working mode during a second time period, and generates a second plasma active gas in the second working mode; and the first time period is before the second time period, and alternatively, the first time period is after the second time period, such that a disinfection object in the disinfection chamber is disinfected. The plasma generating unit in the present disclosure is a simple device, and only one discharge module is used for generating different plasma active gases.

### CROSS-REFERENCE TO RELATED APPLICATIONS

This application claims priority to Chinese Patent Application No. 202310875786.0, filed on Jul. 17, 2023, the content of which is incorporated herein by reference in its entirety.

### TECHNICAL FIELD

The present disclosure relates to the technical field of normal pressure cold plasma application, and in particular to a plasma-activated gas disinfection device and a plasma-activated gas disinfection method.

### BACKGROUND

Currently, common disinfection and sterilization technologies on the market mainly include ultraviolet irradiation sterilization, chemical sterilization, pre-vacuum autoclave sterilization, etc. However, the technologies all have their own shortcomings. The ultraviolet sterilization technology has a dead zone and is only suitable for surface sterilization of an object. The chemical sterilization technology is likely to generate toxic gases when used, which is irritating and highly corrosive to an operator. The pre-vacuum autoclave sterilization technology has a complicated working procedure and is only suitable for the instruments that are resistant to high pressure, high temperature and high humidity. As a new mode of disinfection and sterilization, the application of plasma in the field of disinfection and sterilization has a number of advantages. Plasma is a quasi-electrically neutral macroscopic system containing numerous unbound charged particles and is the fourth state of matter existence. Atmospheric cold plasma, a form of plasma, is generally non-thermal equilibrium plasma with an electron temperature greater than 10,000° C. and a gas temperature close to a room temperature. A plasma active gas produced by atmospheric cold plasma contains a variety of reactive oxygen and nitrogen species (RONS), such as O₃, NO, N₂O₅, NO₃, etc., which can efficiently inactivate various bacteria, fungi and viruses and has a broad-spectrum antimicrobial property and a temperature close to room temperature, so it can be used for sterilization of non-heat-resistant materials. Atmospheric cold plasma is suitable for the field of disinfection and sterilization since as a new mode of sterilization with high efficiency, wide applicability and high safety due to the gas sterilization, it does not leave any dead space and facilitates batch processing and is safer and easier to operate compared with chemical sterilization.

However, there are still technical difficulties in plasma disinfection, first of all, after plasma disinfection, there are residual substances, such as NO₂, O₃ gas, which is harmful to human health. Secondly, the effect of air plasma for batch disinfection needs to be improved, in particular, there is still a gap in the technology of producing high-valence nitrogen oxides by using a plasma generator for effective disinfection.

### SUMMARY

An objective of the present disclosure is to provide a plasma-activated gas disinfection device and a plasma-activated gas disinfection method which can reduce a size of a disinfection device, improve a disinfection effect and reduce residual byproducts after disinfection.

In order to solve the above problems, the present disclosure provides a plasma-activated gas disinfection device, including a plasma generating unit, a disinfection chamber, an air pump and a fluid control module, where the plasma generating unit, the disinfection chamber, the air pump and the fluid control module form a first loop; the plasma generating unit works in a first working mode during a first time period, and generates a first plasma active gas in the first working mode; the plasma generating unit works in a second working mode during a second time period, and generates a second plasma active gas in the second working mode; and the first plasma active gas and the second plasma active gas are circulated in the first loop, where the first time period is before the second time period, and alternatively, the first time period is after the second time period, such that a disinfection object in the disinfection chamber is disinfected.

Preferably, the plasma-activated gas disinfection device further includes a degassing unit, where the plasma generating unit, the disinfection chamber, the air pump, the fluid control module and the degassing unit form a second loop; the first plasma active gas and the second plasma active gas are circulated in the second loop in sequence, such that a disinfection working medium is removed from the gases by means of the degassing unit; and the first loop and the second loop are switched by a loop switching device.

Preferably, the plasma generating unit includes a discharge module, and the discharge module is at least one of a coaxial dielectric barrier discharge module, a surface-oriented dielectric barrier discharge module, a flat-plate-type dielectric barrier discharge module and a corona discharge module.

Preferably, the coaxial dielectric barrier discharge module includes a tube, a high-voltage electrode and a low-voltage electrode, the high-voltage electrode is arranged in the tube, two ends of the high-voltage electrode are respectively connected to two ends of the tube by means of first fixing components, a gap is provided between the high-voltage electrode and the tube, the first fixing component at one end is provided with a gas inlet/outlet of the discharge module, correspondingly, the first fixing component at the other end is provided with a discharge module gas outlet/inlet, the low-voltage electrode is arrange on a peripheral wall of the tube, and the gas inlet/outlet is in communication with the gas outlet/inlet through the gap.

Preferably, the plasma generating unit includes a temperature adjusting module, and the temperature adjusting module is at least one of a semiconductor refrigeration module, a water cooling module and a heating module.

Preferably, in the first working mode, a proportion of nitrogen oxides in a total concentration of active substances in the first plasma active gas is greater than 80%; in the second working mode, a proportion of ozone in the total concentration of active substances in the second plasma active gas is greater than 50%; and the first working mode and the second working mode are switched by means of at least one of power adjustment of the discharge module, flow rate adjustment of the fluid control module, and temperature adjustment of the temperature adjusting module.

Preferably, the degassing unit includes a first plasma active gas removing module and a second plasma active gas removing module, and the first plasma active gas removing module includes a solution for removing the first plasma active gas, and is configured to remove nitrogen oxides from the plasma active gas; and the second plasma active gas removing module includes at least one of a catalyst component for removing the first plasma active gas, and a heating component, and is configured to remove ozone from the plasma active gas.

Preferably, the amount of total nitrogen oxides in the first plasma active gas generated in the first time period under the first working mode is V1, the amount of ozone in the second plasma active gas generated in the second time period under the second working mode is V2, and V1 and V2 satisfy: V2≥2V1.

Preferably, a disinfection method using the plasma-activated gas disinfection device according to any one of the above items includes:
controlling a loop switching device to switch to a first loop, and setting a working mode of a plasma generating unit as a first working mode, such that the plasma generating unit generates a first plasma active gas in a first time period, where the first plasma active gas is circulated in the first loop, to disinfect a disinfection object in a disinfection chamber;
setting the working mode of the plasma generating unit as a second working mode, such that the plasma generating unit generates a second plasma active gas in a second time period, where the second plasma active gas is circulated in the first loop, to disinfect the disinfection object in the disinfection chamber; and
controlling the loop switching device to switch to a second loop after a preset working duration, such that the first plasma active gas and the second plasma active gas are circulated in the second loop, and removing the first plasma active gas and the second plasma active gas from gas by means of the degassing unit; where the first time period is before the second time period, and alternatively, the first time period is after the second time period.

Preferably, the first time period and the second time period satisfy: 1:9≤T1:T2≤5:5, where T1 represents the first time period, and T2 represents the second time period.

The above-mentioned technical solution in the present disclosure has the following beneficial effects.

The plasma generating unit in the present disclosure is a simple device, and the same component generates different plasma active gases according to different working modes, and works in a nitrogen oxide mode and an ozone mode in separated time intervals, such that a yield of high-valence nitrogen oxides can be effectively improved, a yield of ozone is limited, a disinfection effect of the activated gas is effectively enhanced, and high efficiency disinfection and sterilization in the chamber is achieved. The plasma generating unit is a device simple, only one discharge module is used, the cost is saved, the miniaturization of the device is achieved, and the cost is low. A raw material is only air, and residual byproducts such as O₃, etc. after disinfection can be removed only by simple water washing and heating, such that the present disclosure has the advantages of green, low cost and no emission.

### BRIEF DESCRIPTION OF THE DRAWINGS

FIG. 1 is a schematic diagram of an overall structure of an example of the present disclosure.

FIG. 2 is a schematic structural diagram of a discharge module according to an example of the present disclosure.

FIG. 3 is a comparison diagram of sterilization effects of an example of the present disclosure.

FIG. 4 is a diagram illustrating an effect of a degassing unit on removing an ozone gas according to an example of the present disclosure.

FIG. 5 is a diagram illustrating an effect of a degassing unit on removing nitrogen oxides according to an example of the present disclosure.

FIG. 6 is a flowchart of a disinfection method according to an example of the present disclosure.

### DETAILED DESCRIPTION OF THE EMBODIMENTS

In order to make the objectives, technical solutions and advantages of the present disclosure clearer, the present disclosure will be described in further detail below in conjunction with the specific embodiments with reference to the accompanying drawings. It should be understood that these descriptions are merely illustrative and not intended to limit the scope of the present disclosure. Moreover, in the following explanation, descriptions of well-known structures and techniques are omitted to avoid unnecessarily obscuring the concepts of the present disclosure.

A schematic diagram of a layer structure accord to an example of the present disclosure is shown in the drawings. The drawings are not drawn to scale, with certain details exaggerated and certain details omitted for purposes of clarity. Shapes of various regions and layers as well as relative sizes and positional relations between them shown in the figures are merely illustrative. In practice, there may be deviations due to manufacturing tolerances or technical limitations, and those skilled in the art may additionally design regions/layers with different shapes, sizes and relative positions according to actual needs.

Apparently, the examples described are merely some examples rather than all examples of the present disclosure. Based on the examples of the present disclosure, all other examples acquired by those of ordinary skill in the art without making creative efforts fall within the scope of protection of the present disclosure.

In the description of the present disclosure, it should be noted that the terms “first”, “second”, “third”, etc. are merely for descriptions and may not be understood as indication or implication of relative importance.

### Example 1

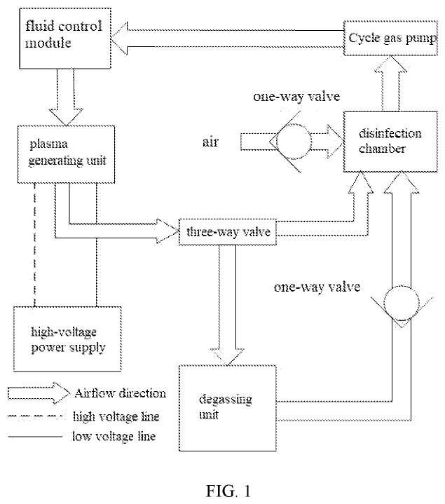

A plasma-activated gas disinfection device is provided. FIG. 1 is a schematic diagram of an overall structure of an example of the present disclosure. As shown in FIG. 1, the plasma-activated gas disinfection device includes a plasma generating unit, a disinfection chamber, an air pump and a fluid control module. The plasma generating unit, the disinfection chamber, the air pump and the fluid control module form a first loop. The plasma generating unit works in a first working mode during a first time period, and generates a first plasma active gas in the first working mode. The plasma generating unit works in a second working mode during a second time period, and generates a second plasma active gas in the second working mode. The first time period is before the second time period, alternatively, the first time period is after the second time period, the first plasma active gas and the second plasma active gas are circulated in the first loop, such that a disinfection object in the disinfection chamber is disinfected. A connection structure of each unit in the first loop is not limited herein. The units may be connected by means of a straight pipe, may be connected by means of a bent pipe, to form the first loop, or may be directly connected without a pipe. A specific structure of the disinfection chamber is not limited, and may be cuboid, cylindrical, or irregular. The disinfection chamber is used for storing an article to be disinfected and fully mixing plasma activation gases in different modes. A specific specification of the air pump is not limited, the air pump is used for driving the gas to be circulated in the loop, and the plasma generating unit is used for generating the plasma activated gases. The plasma generating unit generates cold plasma to activate the gas, and a working gas is air. Alternatively, a disinfection device gas inlet is arranged in the disinfection chamber, and further, a one-way valve is arranged. When the gas in the disinfection chamber undergoes cracking and recombination reaction after being treated by discharge plasma, an internal pressure is lower than an atmospheric pressure, and the one-way valve may keep the internal pressure of the disinfection chamber as the standard atmospheric pressure. The fluid control module monitors a gas flow rate in the first loop. A specific content of the fluid control module is not limited herein, and alternatively, is a single chip microcomputer, a digital signal processor, and an application specific integrated circuit (ASIC). Alternatively, in the example, the fluid control module is a single chip microcomputer with a model of STM32F103RCT6. A method for switching the working mode of the plasma generating unit from the first working mode to the second working mode includes: increase the gas flow rate from a first flow rate threshold to a second flow rate threshold by means of the fluid control module. Specific contents of the first flow rate threshold and the second flow rate threshold are not limited herein, and it is only limited that a maximum value of the first flow rate threshold is less than or equal to a minimum value of the second flow rate threshold. When power of the discharge module is fixed, the working mode of the plasma generating unit may be switched from the first working mode to the second working mode by increasing the flow rate of the gas. The method is simple, the cost is low, and popularization is facilitated.

In an example of the present disclosure, further, the plasma-activated gas disinfection device further includes a degassing unit. The plasma generating unit, the disinfection chamber, the air pump, the fluid control module and the degassing unit form a second loop; the first plasma active gas and the second plasma active gas are circulated in the second loop, such that a disinfection working medium is removed from the gases by means of the degassing unit. The first loop and the second loop are switched by a loop switching device. A specific content of the degassing unit is not limited herein. Further, the degassing unit includes a disinfection device gas outlet, which is a gas outlet of the entire device. The degassing unit is used for eliminating a disinfection working medium in the plasma activated gases and reducing environmental pollution. A specific content of the loop switching device is not limited herein, and alternatively, may be a three-way valve, or two single valves, etc. In this example, the loop switching device may be a three-way valve for switch.

According to the example, the device is simple, only one discharge module is used to switch two plasma discharge modes, the cost is saved, and the miniaturization of the device is achieved. Compared with a single mode, in this example, the nitrogen oxide mode and the ozone mode are time-sharing combined by adjusting the working mode, and more high-valence nitrogen oxides are generated after the plasma activated gases of different working modes are mixed, such that a better sterilization effect is obtained. In the example, tail gas treatment after disinfection is considered, the degassing unit is added, residual disinfection working medium after disinfection is removed through the second loop, and a generated harmful gas is removed, such that a disinfection process is cleaner, and environmental pollution is reduced.

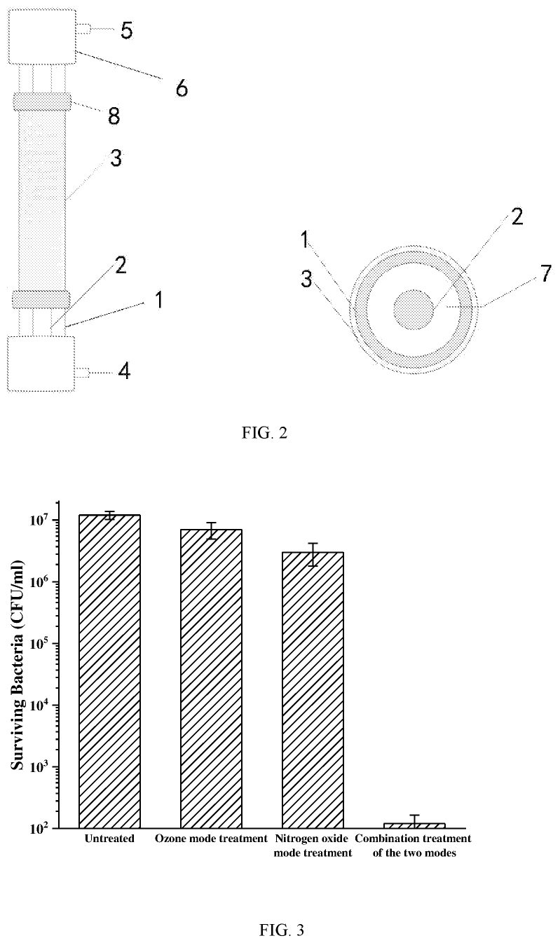

In an example of the present disclosure, further, the plasma generating unit includes a discharge module, and the discharge module is at least one of a coaxial dielectric barrier discharge module, a surface-oriented dielectric barrier discharge module, a flat-plate-type dielectric barrier discharge module and a corona discharge module. FIG. 2 is a schematic structural diagram of a discharge module according to an example of the present disclosure. The discharge module is at least one of a coaxial dielectric barrier discharge module, a surface-oriented dielectric barrier discharge module, a flat-plate-type dielectric barrier discharge module and a corona discharge module. As shown in FIG. 2, the coaxial dielectric barrier discharge module includes a tube 1, a high-voltage electrode 2 and a low-voltage electrode 3, the high-voltage electrode 2 is arranged in the tube 1, two ends of the high-voltage electrode 2 are respectively connected to two ends of the tube 1 by means of first fixing components 6, a gap 7 is provided between the high-voltage electrode 2 and the tube 1, the first fixing component at one end is provided with a gas inlet/outlet 4 of the discharge module, correspondingly, the first fixing component at the other end is provided with a discharge module gas outlet/inlet 5, the low-voltage electrode 3 is arrange on a peripheral wall of the tube 1, and the gas inlet/outlet 4 is in communication with the gas outlet/inlet 5 through the gap 7. A specific material of the tube 1 is not limited herein, and alternatively, may be a silicon substrate, quartz, etc. Alternatively, in this example, the tube 1 is made from quartz. A specific material and structure of the high-voltage electrode 2 are not limited herein, and alternatively, the high-voltage electrode may be made from a metal material, such as gold, silver, copper, iron, nickel alloy, etc. Alternatively, in this example, the high-voltage electrode is made from stainless steel, and may have a cylindrical structure. A relation between axial lengths of the tube and the high-voltage electrode is not limited herein, and alternatively, the axial lengths are the same. The gap 7 is provided between the high-voltage electrode 2 and the tube 1. A specific size of the gap 7 is not limited herein, and it is not limited whether the gap 7 is uniform. In this example, the gap 7 is uniform. A specific material and structure of the low-voltage electrode 3 are not limited herein, and alternatively, the low-voltage electrode may be made from a metal material, such as gold, silver, copper, iron, nickel alloy, etc. Alternatively, in this example, the low-voltage electrode is made from copper, and may have an annular structure and further have a mesh structure. In this example, the low-voltage electrode have a copper mesh ring structure and is fixed by the second fixing component 8. A relation between axial lengths of the low-voltage electrode and the tube is not limited herein. Alternatively, in this example, the axial length of the low-voltage electrode is less than the axial length of the tube. A specific material and structure of the first fixing component 6 are not limited herein. Alternatively, the first fixing component may have a square, circular, or other barrel-shaped structure. A specific material and structure of the second fixing component 8 are not limited herein. Alternatively, the second fixing component may be a screw, a buckle, etc. In this example, alternatively, the second fixing component is an annular copper hoop. The two ends of the low-voltage electrode 3 are fixed to the tube 1 through circular copper hoops. Alternating current high-voltage excitation is applied between the stainless steel high-voltage electrode and the mesh low-voltage electrode, the discharge module may generate atmospheric cold plasma after dielectric barrier discharge, and the cold plasma may activate a gas to be activated in the gap, so as to generate a plasma activated gas. A method for switching the working mode of the plasma generating unit from the first working mode to the second working mode includes: lower the power of the discharge module from a first power threshold to a second power threshold by means of a high-voltage power supply. The first power threshold and the second power threshold are not specifically limited herein, and it is only limited that a maximum value of the second power threshold is less than or equal to a minimum value of the first power threshold. The discharge generated by the discharge module of the example is more stable, a better discharge effect is obtained, and an industrial utilization rate of the discharge module is improved.

In an example of the present disclosure, further, the plasma generating unit includes a temperature adjusting module, and the temperature adjusting module is at least one of a semiconductor refrigeration module, a water cooling module and a heating module. Alternatively, in this example, the temperature adjusting module is a semiconductor refrigeration module. A method for switching the working mode of the plasma generation unit from the first working mode to the second working mode includes: lower a first temperature threshold to a second temperature threshold by means of the temperature adjusting module. Specific contents of the first temperature threshold and the second temperature threshold are not limited herein, and it is only limited that a minimum value of the first temperature threshold is greater than or equal to a maximum value of the second temperature threshold. By lowering the temperature, the working mode of the plasma generating unit may be switched from the first working mode to the second working mode when the power of the discharge module is fixed. The method is simple, the cost is low, and the popularization is facilitated.

In an example of the present disclosure, further, in the first working mode, a proportion of nitrogen oxides in a total concentration of active substances in the first plasma active gas is greater than 80%; in the second working mode, a proportion of ozone in the total concentration of active substances in the second plasma active gas is greater than 50%; and the first working mode and the second working mode are switched by means of at least one of power adjustment of the discharge module, flow rate adjustment of the fluid control module, and temperature adjustment of the temperature adjusting module. Specifically, a method for switching the working mode of the plasma generating unit from the first working mode to the second working mode at least includes one of the following steps: increase the power of the discharge module from the first power threshold to the second power threshold by means of the high-voltage power supply; increase the gas flow rate from the first flow rate threshold to the second flow rate threshold by means of the fluid control module; and lower the first temperature threshold to the second temperature thresholds by means of the temperature regulating module. Further, a method for switching the working mode of the plasma generating unit from the first working mode to the second working mode may include one of the following steps: keep the gas flow rate and the temperature unchanged, and lower the power of the discharge module from the first power threshold to the second power threshold by means of the high-voltage power supply; keep the discharge power and the temperature unchanged, and increase the gas flow rate from the first flow rate threshold to the second flow rate threshold by means of the fluid control module; and keep the discharge power and the gas flow rate unchanged, and lower the first temperature threshold to the second temperature threshold by means of the temperature regulating module. Alternatively, the method includes: adjust the gas flow rate and the discharge power, to adjust the temperature, so as to lower the first temperature threshold to the second temperature threshold; or adjust the gas flow rate, the discharge power and the temperature, to adjust the temperature, so as to lower the first temperature threshold the second temperature threshold.

Alternatively, in this example, the first working mode is switched to the second working mode, in the first working mode, the temperature of the plasma generating unit is controlled to be not less than 60° C., and a nitrogen oxide yield accounts for greater than 80% of a gas phase product of the first plasma active gas. When the first working mode is switched to the second working mode, a maximum temperature of a plasma region of the plasma generating unit is controlled to be reduced to below 60° C. within 1 minute, further, a stable working temperature of the plasma generating unit is not greater than 55° C., and an ozone yield accounts for greater than 50% of all plasma gas phase products. A specific manner of temperature adjustment is not limited herein. Cooling may be performed by means of the temperature adjusting module, cooling may be performed by increasing the flow rate of the fluid control module, cooling may be performed by means of the power of the discharge module, and alternatively, cooling may be performed through any combination.

Alternatively, in this example, the power of the discharge module and the temperature of the plasma generating unit are fixed; or the power of the discharge module is fixed, and the temperature is not adjusted since a change in flow rate may also cause a change in temperature. The temperature is not limited herein. When the gas flow rate is controlled at 1 L/min, main products of the plasma activated gas are nitrogen oxides (such as NO and NO₂). When the gas flow rate is controlled at 3 L/min, main products of the plasma activated gas are ozone. The working mode of the plasma generating unit is switched by adjusting and controlling the gas flow rate.

Alternatively, in this example, the gas flow rate and the temperature of the plasma generating unit are fixed; or the gas flow rate is fixed, and the temperature is not adjusted since a change of the discharge power may also cause a change in temperature. The temperature is not limited herein. When the discharge power is controlled at 40 W, main products of the plasma activated gas are nitrogen oxides (such as NO and NO₂). When the discharge power is controlled at 20 W, main products of the plasma activated gas are ozone. The working mode of the plasma generating unit is switched by adjusting the discharge power.

In the example, the working mode of the plasma generating unit is adjusted by controlling one or any combination of the gas flow rate, the discharge power and the working temperature of the plasma generating unit, such that a disinfection efficiency is improved, the cost is low, the realization is easy, and popularization is facilitated.

In an example of the present disclosure, further, the degassing unit includes a first plasma active gas removing module and a second plasma active gas removing module, and the first plasma active gas removing module includes a solution for removing the first plasma active gas, and is configured to remove nitrogen oxides from the plasma active gas; and the second plasma active gas removing module includes at least one of a catalyst component for removing the first plasma active gas, and a heating component, and is configured to remove ozone from the plasma active gas. Specific contents of the first plasma active gas removing module and the second plasma active gas removing module are not limited herein. Alternatively, the first plasma active gas removing module includes a gas washing chamber, and the gas washing chamber includes a solution for removing high-valence nitrogen oxides. The solution is water or alkaline solution. A volume and placement mode of the solution are not limited herein, and may be spread on a bottom surface of the degassing unit in a larger area or concentrated on a part of the bottom surface of the degassing unit. A specific content of the alkaline solution is not limited herein, and alternatively, may be sodium hydroxide, potassium hydroxide, sodium carbonate, sodium bicarbonate, ammonia water, etc. In this example, alternatively, the solution may be 0.2 mol/L sodium hydroxide solution with a volume of 500 ml. The second plasma active gas removing module includes an ozone quenching module, and the ozone quenching module is used for further removing ozone. The ozone quenching module is at least one of a catalyst component and a heating component. The catalyst component includes a drying tube and a catalyst carrier. The drying tube is used for removing water vapor carried out from the gas washing chamber, to prevent the service life of the catalyst from being reduced. The catalyst carrier has a porous structure, to make a contact area between the plasma activated gas and the catalyst large. The heating component ozone quenching module includes a heating pipe for raising the temperature of the plasma activated gas to 60±5° C., to quench the ozone. The degassing unit may purify different disinfection gases in a targeted manner, so as to reduce environmental pollution.

In an example of the present disclosure, further, the amount (mol) of total nitrogen oxides in the first plasma active gas generated in the first time period in the first working mode is V1, the amount (mol) of ozone in the second plasma active gas generated in the second time period under the second working mode is V2, and V1 and V2 satisfy: V2≥2V1.

The yield of the second plasma active gas generated in the second working duration in the second working mode is greater than or equal to twice the yield of the first plasma active gas generated in the first working duration in the first working mode. The plasma generating unit firstly works in the first working mode, that is, the nitrogen oxide mode and then works in the second working mode, that is, the ozone mode in different time intervals. When the plasma generating unit stops, a large amount of ozone needs to exist, and all NO and NO₂ generated in all nitrogen oxide modes are oxidized into higher-valence nitrogen oxides, such as N₂O₅ and NO₃, which has a better disinfection effect.

### Example 2

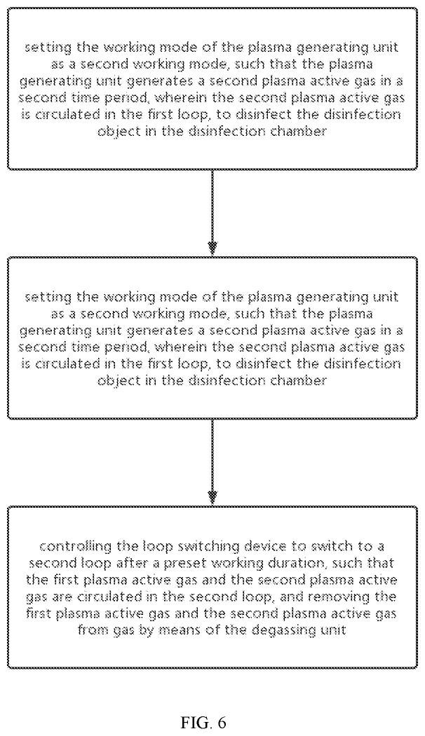

According to a disinfection method using the plasma-activated gas disinfection device according to any one of the above items, FIG. 6 is a flowchart of the disinfection method according to an example of the present disclosure. As shown in FIG. 6, the method includes:
controlling a loop switching device to switch to a first loop, and setting a working mode of a plasma generating unit as a first working mode, such that the plasma generating unit generates a first plasma active gas in a first time period, and the first plasma active gas is circulated in the first loop, to disinfect a disinfection object in a disinfection chamber. Herein, a working condition of the plasma generating unit is controlled to satisfy at least one of a first flow rate threshold, a first power threshold and a first temperature threshold, that is, to fix power of a discharge module and adjust a gas flow rate to the first flow rate threshold, alternatively, to fix the power of the discharge module and adjust a temperature to the first temperature threshold, and alternatively, to adjust the power of the discharge module to the first power threshold. Alternatively, in this example, the power of the discharge module is fixed at 40 W. When the gas flow rate is controlled at 1 L/min, main products of the plasma activated gas are nitrogen oxides (such as NO and NO₂). When the gas flow rate is controlled at 3 L/min, main products of the plasma activated gas are ozone. The working mode of the plasma generating unit is switched by adjusting and controlling the gas flow rate. A sequence of the first time period and the second time period is not limited herein. Alternatively, in this example, the first time period is prior to the second time period. Alternatively, the first working duration of the first working mode accounts for 10%-50% of the first time period and the second time period of the plasma generating unit.

The method further includes: setting the working mode of the plasma generating unit to a second working mode, and controlling the working condition of the plasma generating unit to satisfy at least one of a second flow rate threshold, a second power threshold and a second temperature threshold, such that the plasma generating unit generates a second plasma active gas within a second time period, and the second plasma active gas is circulated in the first loop, to disinfect the disinfection object in the disinfection chamber. The nitrogen oxide mode and the ozone mode are time-sharing combined by adjusting the working mode, and more high-valence nitrogen oxides are generated after the plasma activated gases of different working modes are mixed, such that a better sterilization effect is obtained. A specific content of the second time period is not limited herein, and alternatively, the second time period accounts for 50%-90% of the first time period and the second time period of working of the plasma generating unit. The nitrogen oxide mode and the ozone mode are mixed in a time-sharing manner regardless of the sequence of working of the nitrogen oxide mode and the ozone mode. A total working duration is not limited herein. Alternatively, in this example, the total duration of the first time period and the second time period of the plasma generating unit is set to be 5 min.

The first time period and the second time period satisfy: 1:9≤T1:T2≤5:5, where T1 represents the first time period, T2 represents the second time period, and an order of the first time period and the second time period is not limited herein.

The sequence of the first time period and the second time period is not limited. The first time period is before the second time period, and alternatively, the first time period is after the second time period. That is to say, the second working mode may be set first, such that the plasma generating unit generates the second plasma active gas in the second time period, and then the first working mode may be set, such that the plasma generating unit generates the first plasma active gas in the first time period. Alternatively, in this example, the first time period is before the second time period.

A specific time for disinfecting an article is not limited herein. Alternatively, in this example, the disinfection time is 5 min. Under the same discharge power, the entire process time is fixed to be 10 min, where 5 min is the working time of the plasma generation unit, and 5 min is the disinfection time after the plasma generating unit is turned off. Comparing sterilization effects of ozone mode treatment, nitrogen oxide mode treatment and time-sharing combination treatment of the two modes, in this example, the stable first working mode is the nitrogen oxide mode when the flow rate is set to be 1 L/min, and the stable second working mode is the ozone mode when the flow rate is set to be 3 L/min. Three experimental groups are set up, which are 5 min nitrogen oxide mode, 5 min ozone mode, and a time-sharing combination mode of 2.5 min nitrogen oxide mode and 2.5 min ozone mode. 10 μl of Staphylococcus aureus suspension (concentration OD600=10) is evenly spread on a slide with a size of 10 mm*10 mm, and the slide is dried in the shade and put into the disinfection chamber for disinfection treatment. Results are shown in FIG. 3. In FIG. 3, a sterilization effect of a time-sharing treatment manner of the two modes is 1-2 orders of magnitude lower than that of a single mode of sterilization, and the time-sharing treatment manner of the two modes has a more efficient sterilization effect.

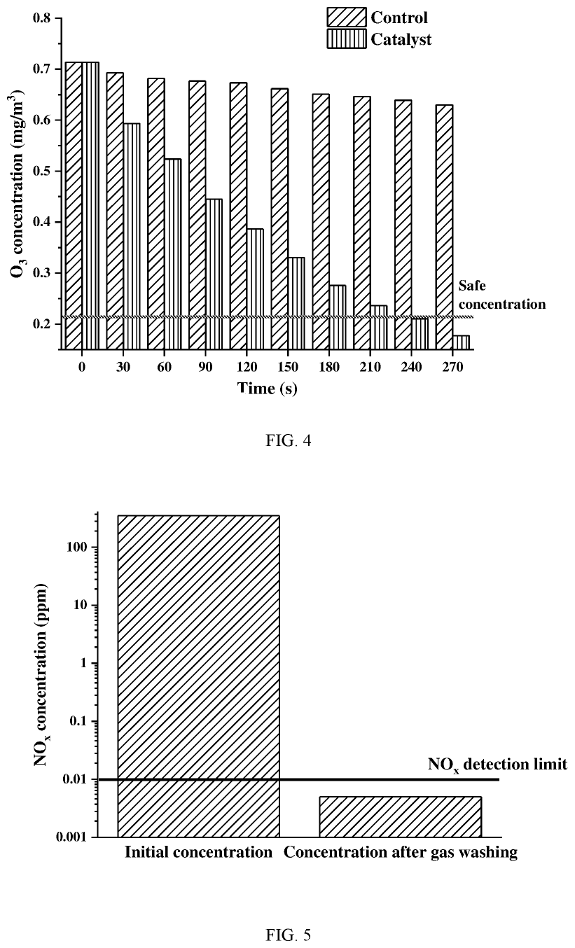

The loop switching device is controlled to switch to a second loop after a preset working duration, such that the first plasma active gas and the second plasma active gas are circulated in the second loop, and the first plasma active gas and the second plasma active gas are removed from gas by means of the degassing unit. The sequence of operation is to remove nitrogen oxides first and then ozone. A preset working duration is not limited herein. Different working durations can be set according to different disinfection objects. The working duration of the degassing unit is also not limited herein. After the degassing unit eliminates the disinfection gas generated by the plasma generating unit, the gas is discharged from the disinfection device gas outlet. A degassing time is not limited herein. In this example, a degassing effect of setting the degassing time to be 5 min is detected. FIG. 4 is a diagram illustrating an effect of the degassing unit on removing an ozone gas according to an example of the present disclosure. FIG. 5 is a diagram illustrating an effect of the degassing unit on removing nitrogen oxides according to an example of the present disclosure. First, a removal efficiency of ozone is shown in FIG. 4. As measured by a gas measurement module, an initial ozone concentration after disinfection is 0.71 mg/m³. A decomposition rate of ozone is slow without a degassing module. It is rapid when adding an ozone quenching module with a porous manganese dioxide catalyst, and the concentration is lower than the national safety standard of 2.14 mg/m³ at 90 s. Second, a removal efficiency of nitrogen oxides is shown in FIG. 5. An initial NOₓ concentration is 350 ppm measured by the gas measurement module. Through a gas washing bottle with 500 ml of a 0.2 mol/L sodium hydroxide solution, NOₓ is lower than a measurement limit of 0.1 ppm only through single absorption. The gas in the chamber can be ventilated for many times within 5 min, such that NOₓ is almost completely removed. The above experimental results confirm that the disinfection working medium (NOₓ, O₃) can satisfy a safety emission standard after 5 min of removal by the degassing module.

In the example, switching of two working modes of the plasma generating unit is implemented by means of at least one of discharge power control, fluid control and temperature control, which is beneficial to save the cost and achieve the miniaturization of the device. Compared with a single mode, time-sharing combination of the nitrogen oxide mode and the ozone mode is achieved, the plasma nitrogen-oxygen mode and the ozone mode can be combined in the same treatment time, and more high-valence nitrogen oxides are generated after plasma activated gases in different modes are mixed, such that a better sterilization effect is obtained. Moreover, the above implementation considers tail gas treatment after disinfection, the degassing unit is added to remove residual ozone and high-valence nitrogen oxides (such as NO₃ and N₂O₅) after disinfection through the second loop, so as to remove the generated harmful gases and make the disinfection process cleaner.

It is to be understood that the above specific embodiments in the present disclosure are for illustrative description or explanation of principles of the present disclosure only and not limitation of the present disclosure. Therefore, any modifications, equivalent substitutions, improvements, etc. without departing from the spirit and scope of the present disclosure are intended to be included within the scope of protection of the present disclosure. Furthermore, the appended claims of the present disclosure intends to cover all variations and modifications that fall within the scope and boundaries of the appended claims or the equivalents of such scopes and boundaries.

What is claimed is:

1. A plasma-activated gas disinfection device, comprising a plasma generating unit, a disinfection chamber, an air pump and a fluid control module, wherein
the plasma generating unit, the disinfection chamber, the air pump and the fluid control module form a first loop;
the plasma generating unit works in a first working mode during a first time period, and generates a first plasma active gas in the first working mode; the plasma generating unit works in a second working mode during a second time period, and generates a second plasma active gas in the second working mode; and the first plasma active gas and the second plasma active gas are circulated in the first loop, wherein the first time period is before or after the second time period, such that a disinfection object in the disinfection chamber is disinfected.

2. The plasma-activated gas disinfection device according to claim 1, further comprising a degassing unit, wherein
the plasma generating unit, the disinfection chamber, the air pump, the fluid control module and the degassing unit form a second loop; the first plasma active gas and the second plasma active gas are circulated in the second loop in sequence, such that a disinfection working medium is removed from the gases by means of the degassing unit; and
the first loop and the second loop are switched by a loop switching device.

3. The plasma-activated gas disinfection device according to claim 1, wherein the plasma generating unit comprises a discharge module, and the discharge module is at least one of a coaxial dielectric barrier discharge module, a surface-oriented dielectric barrier discharge module, a flat-plate-type dielectric barrier discharge module and a corona discharge module.

4. The plasma-activated gas disinfection device according to claim 3, wherein the coaxial dielectric barrier discharge module comprises a tube, a high-voltage electrode and a low-voltage electrode, the high-voltage electrode is arranged in the tube, two ends of the high-voltage electrode are respectively connected to two ends of the tube by means of first fixing components, a gap is provided between the high-voltage electrode and the tube, the first fixing component at one end is provided with a gas inlet/outlet of the discharge module, correspondingly, the first fixing component at another end is provided with a gas outlet/inlet of the discharge module, the low-voltage electrode is arrange on a peripheral wall of the tube, and the gas inlet/outlet is in communication with the gas outlet/inlet through the gap.

5. The plasma-activated gas disinfection device according to claim 1, wherein the plasma generating unit comprises a temperature adjusting module, and the temperature adjusting module is at least one of a semiconductor refrigeration module, a water cooling module and a heating module.

6. The plasma-activated gas disinfection device according to claim 5, wherein in the first working mode, a proportion of nitrogen oxides in a total concentration of active substances in the first plasma active gas is greater than 80%; in the second working mode, a proportion of ozone in the total concentration of active substances in the second plasma active gas is greater than 50%; and
the first working mode and the second working mode are switched by means of at least one of power adjustment of the discharge module, flow rate adjustment of the fluid control module, and temperature adjustment of the temperature adjusting module.

7. The plasma-activated gas disinfection device according to claim 2, wherein the degassing unit comprises a first plasma active gas removing module and a second plasma active gas removing module, and the first plasma active gas removing module comprises a solution for removing the first plasma active gas, and is configured to remove nitrogen oxides from the plasma active gas; and the second plasma active gas removing module comprises at least one of a catalyst component for removing the first plasma active gas, and a heating component, and is configured to remove ozone from the plasma active gas.

8. The plasma-activated gas disinfection device according to claim 1, wherein an amount of total nitrogen oxides in the first plasma active gas generated in the first time period under the first working mode is V1, an amount of ozone in the second plasma active gas generated in the second time period under the second working mode is V2, and V1 and V2 satisfy:
$$V 2 \geq 2 V 1.$$

9. A disinfection method using the plasma-activated gas disinfection device according to claim 1, comprising:
controlling a loop switching device to switch to a first loop, and setting a working mode of a plasma generating unit as a first working mode, such that the plasma generating unit generates a first plasma active gas in a first time period, wherein the first plasma active gas is circulated in the first loop, to disinfect a disinfection object in a disinfection chamber;
setting the working mode of the plasma generating unit as a second working mode, such that the plasma generating unit generates a second plasma active gas in a second time period, wherein the second plasma active gas is circulated in the first loop, to disinfect the disinfection object in the disinfection chamber; and
controlling the loop switching device to switch to a second loop after a preset working duration, such that the first plasma active gas and the second plasma active gas are circulated in the second loop, and removing the first plasma active gas and the second plasma active gas from gas by means of the degassing unit; wherein
the first time period is before or after the second time period.

10. The disinfection method according to claim 9, wherein the first time period and the second time period satisfy:
$$1 : 9 \leq T 1 : \qquad T 2 \leq 5 : 5 ,$$
wherein T1 represents the first time period, and T2 represents the second time period.
